(19) United States
(12) Patent Application Publication
(10) Pub. No.: US 2006/0167437 A1
(43) Pub. Date: Jul. 27, 2006

(54) Method and apparatus for intra aortic substance delivery to a branch vessel

(75) Inventor: Aurelio Valencia, East Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 11/303,554

(22) Filed: Dec. 16, 2005

Related U.S. Application Data
(63) Continuation of application No. PCT/US04/08571, filed on Mar. 19, 2004.
(60) Provisional application No. 60/508,751, filed on Oct. 2, 2003.
(60) Provisional application No. 60/502,389, filed on Sep. 13, 2003.
(60) Provisional application No. 60/479,329, filed on Jun. 17, 2003.

(51) Int. Cl. A61M 25/00

(52) U.S. Cl. 604/523; 604/104

(57) ABSTRACT

A renal flow system injects a volume of fluid agent into a location within an abdominal aorta in a manner that flows bilaterally into each of two renal arteries via their respectively spaced ostia along the abdominal aorta wall. A local injection assembly (100) includes two injection members (104, 106), each having an injection port (112) that couples to a source of fluid agent externally of the patient. The injection ports may be positioned within an outer region of blood flow along the abdominal aorta wall perfusing the two renal arteries.

### CROSS-REFERENCE TO RELATED APPLICATIONS

This application claims priority from U.S. provisional application Ser. No. 60/508,751 filed on Oct. 2, 2003, incorporated herein by reference in its entirety.

This application claims priority from, and is a continuation-in-part of, PCT International Application Serial No. PCT/US2003/029995 filed on Sep. 22, 2003, which designates the U.S., incorporated herein by reference in its entirety.

This application claims priority to U.S. provisional application 60/502,389 filed on Sep. 13, 2003, incorporated herein by reference in its entirety.

This application claims priority from U.S. provisional application Ser. No. 60/479,329 filed on Jun. 17, 2003, incorporated herein by reference in its entirety.

This application claims priority from U.S. provisional application Ser. No. 60/412,343 filed on Sep. 20, 2002, incorporated herein by reference in its entirety.

This application claims priority from U.S. provisional application Ser. No. 60/412,476 filed on Sep. 20, 2002, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to medical device systems and methods for intra aortic fluid delivery into regions of the body. More specifically, it is related to intra aortic renal fluid delivery systems and methods.

2. Description of Related Art

Many different medical device systems and methods have been previously disclosed for locally delivering fluids or other agents into various body regions, including body lumens such as vessels, or other body spaces such as organs or heart chambers. Local “fluid” delivery systems may include drugs or other agents, or may even include locally delivering the body's own fluids, such as artificially enhanced blood transport (e.g. either entirely within the body such as directing or shunting blood from one place to another, or in extracorporeal modes such as via external blood pumps etc.). Local “agent” delivery systems are herein generally intended to relate to introduction of a foreign composition as an agent into the body, which may include drug or other useful or active agent, and may be in a fluid form or other form such as gels, solids, powders, gases, etc. It is to be understood that reference to only one of the terms fluid, drug, or agent with respect to local delivery descriptions may be made variously in this disclosure for illustrative purposes, but is not generally intended to be exclusive or omissive of the others; they are to be considered interchangeable where appropriate according to one of ordinary skill unless specifically described to be otherwise.

In general, local agent delivery systems and methods are often used for the benefit of achieving relatively high, localized concentrations of agent where injected within the body in order to maximize the intended effects there and while minimizing unintended peripheral effects of the agent elsewhere in the body. Where a particular dose of a locally delivered agent may be efficacious for an intended local effect, the same dose systemically delivered would be substantially diluted throughout the body before reaching the same location. The agent's intended local effect is equally diluted and efficacy is compromised. Thus systemic agent delivery requires higher dosing to achieve the required localized dose for efficacy, often resulting in compromised safety due to for example systemic reactions or side effects of the agent as it is delivered and processed elsewhere throughout the body other than at the intended target.

Various diagnostic systems and procedures have been developed using local delivery of dye (e.g. radiopaque “contrast” agent) or other diagnostic agents, wherein an external monitoring system is able to gather important physiological information based upon the diagnostic agent's movement or assimilation in the body at the location of delivery and/or at other locations affected by the delivery site. Angiography is one such practice using a hollow, tubular angiography catheter for locally injecting radiopaque dye into a blood chamber or vessel, such as for example coronary arteries in the case of coronary angiography, or in a ventricle in the case of cardiac ventriculography.

Other systems and methods have been disclosed for locally delivering therapeutic agent into a particular body tissue within a patient via a body lumen. For example, angiographic catheters of the type just described above, and other similar tubular delivery catheters, have also been disclosed for use in locally injecting treatment agents through their delivery lumens into such body spaces within the body. More detailed examples of this type include local delivery of thrombolytic drugs such as TPA™, heparin, cumadin, or urokinase into areas of existing clot or thrombogenic implants or vascular injury. In addition, various balloon catheter systems have also been disclosed for local administration of therapeutic agents into target body lumens or spaces, and in particular associated with blood vessels. More specific previously disclosed of this type include balloons with porous or perforated walls that elute drug agents through the balloon wall and into surrounding tissue such as blood vessel walls. Yet further examples for localized delivery of therapeutic agents include various multiple balloon catheters that have spaced balloons that are inflated to engage a lumen or vessel wall in order to isolate the intermediate catheter region from in-flow or out-flow across the balloons. According to these examples, a fluid agent delivery system is often coupled to this intermediate region in order to fill the region with agent such as drug that provides an intended effect at the isolated region between the balloons.

The diagnosis or treatment of many different types of medical conditions associated with various different systems, organs, and tissues, may also benefit from the ability to locally deliver fluids or agents in a controlled manner. In particular, various conditions related to the renal system would benefit a great deal from an ability to locally deliver of therapeutic, prophylactic, or diagnostic agents into the renal arteries.

Acute renal failure (“ARF”) is an abrupt decrease in the kidney's ability to excrete waste from a patient's blood. This change in kidney function may be attributable to many causes. A traumatic event, such as hemorrhage, gastrointestinal fluid loss, or renal fluid loss without proper fluid replacement may cause the patient to go into ARF. Patients may also become vulnerable to ARF after receiving anesthesia, surgery, or α-adrenergic agonists because of related systemic or renal vasoconstriction. Additionally, systemic vasodilation caused by anaphylaxis, and anti-hypertensive drugs, sepsis or drug overdose may also cause ARF because the body's natural defense is to shut down, i.e., vasoconstrict, non-essential organs such as the kidneys. Reduced cardiac output caused by cardiogenic shock, congestive heart failure, pericardial tamponade or massive pulmonary embolism creates an excess of fluid in the body, which can exacerbate congestive heart failure. For example, a reduction in blood flow and blood pressure in the kidneys due to reduced cardiac output can in turn result in the retention of excess fluid in the patient's body, leading, for example, to pulmonary and systemic edema.

Previously known methods of treating ARF, or of treating acute renal insufficiency associated with congestive heart failure (“CHF”), involve administering drugs. Examples of such drugs that have been used for this purpose include, without limitation: vasodilators, including for example papavarine, fenoldopam mesylate, calcium-channel blockers, atrial natriuretic peptide (ANP), acetylcholine, nifedipine, nitroglycerine, nitroprusside, adenosine, dopamine, and theophylline; antioxidants, such as for example acetylcysteine; and diuretics, such as for example mannitol, or furosemide. However, many of these drugs, when administered in systemic doses, have undesirable side effects. Additionally, many of these drugs would not be helpful in treating other causes of ARF. While a septic shock patient with profound systemic vasodilation often has concomitant severe renal vasoconstriction, administering vasodilators to dilate the renal artery to a patient suffering from systemic vasodilation would compound the vasodilation system wide. In addition, for patients with severe CHF (e.g., those awaiting heart transplant), mechanical methods, such as hemodialysis or left ventricular assist devices, may be implemented. Surgical device interventions, such as hemodialysis, however, generally have not been observed to be highly efficacious for long-term management of CHF. Such interventions would also not be appropriate for many patients with strong hearts suffering from ARF.

The renal system in many patients may also suffer from a particular fragility, or otherwise general exposure, to potentially harmful effects of other medical device interventions. For example, the kidneys as one of the body's main blood filtering tools may suffer damage from exposed to high density radiopaque contrast dye, such as during coronary, cardiac, or neuro angiography procedures. One particularly harmful condition known as “radiocontrast nephropathy” or “RCN” is often observed during such procedures, wherein an acute impairment of renal function follows exposure to such radiographic contrast materials, typically resulting in a rise in serum creatinine levels of more than 25% above baseline, or an absolute rise of 0.5 mg/dl within 48 hours. Therefore, in addition to CHF, renal damage associated with RCN is also a frequently observed cause of ARF. In addition, the kidneys' function is directly related to cardiac output and related blood pressure into the renal system. These physiological parameters, as in the case of CHF, may also be significantly compromised during a surgical intervention such as an angioplasty, coronary artery bypass, valve repair or replacement, or other cardiac interventional procedure. Therefore, the various drugs used to treat patients experiencing ARF associated with other conditions such as CHF have also been used to treat patients afflicted with ARF as a result of RCN. Such drugs would also provide substantial benefit for treating or preventing ARF associated with acutely compromised hemodynamics to the renal system, such as during surgical interventions.

There would be great advantage therefore from an ability to locally deliver such drugs into the renal arteries, in particular when delivered contemporaneous with surgical interventions, and in particular contemporaneous with radiocontrast dye delivery. However, many such procedures are done with medical device systems, such as using guiding catheters or angiography catheters having outer dimensions typically ranging between about 4 French to about 12 French, and ranging generally between about 6 French to about 8 French in the case of guide catheter systems for delivering angioplasty or stent devices into the coronary or neurovascular arteries (e.g. carotid arteries). These devices also are most typically delivered to their respective locations for use (e.g. coronary ostia) via a percutaneous, translumenal access in the femoral arteries and retrograde delivery upstream along the aorta past the region of the renal artery ostia. A Seldinger access technique to the femoral artery involves relatively controlled dilation of a puncture hole to minimize the size of the intruding window through the artery wall, and is a preferred method where the profiles of such delivery systems are sufficiently small. Otherwise, for larger systems a “cut-down” technique is used involving a larger, surgically made access window through the artery wall.

Accordingly, an intra aortic renal agent delivery system for contemporaneous use with other retrogradedly delivered medical device systems, such as of the types just described above, would preferably be adapted to allow for such interventional device systems, in particular of the types and dimensions just described, to pass upstream across the renal artery ostia (a) while the agent is being delivered into the renal arteries, and (b) while allowing blood to flow downstream across the renal artery ostia, and (c) in an overall cooperating system that allows for Seldinger femoral artery access. Each one of these features (a), (b), or (c), or any sub-combination thereof, would provide significant value to patient treatment; an intra aortic renal delivery system providing for the combination of all three features is so much the more valuable.

Notwithstanding the clear needs for and benefits that would be gained from such intra aortic drug delivery into the renal system, the ability to do so presents unique challenges as follows.

In one regard, the renal arteries extend from respective ostia along the abdominal aorta that are significantly spaced apart from each other circumferentially around the relatively very large aorta. Often, these renal artery ostia are also spaced from each other longitudinally along the aorta with relative superior and inferior locations. This presents a unique challenge to deliver drugs or other agents into the renal system on the whole, which requires both kidneys to be fed through these separate respective arteries via their uniquely positioned and substantially spaced apart ostia. This becomes particularly important where both kidneys may be equally at risk, or are equally compromised, during an invasive upstream procedure—or, of course, for any other indication where both kidneys require renal drug delivery. Thus, an appropriate intra aortic delivery system for such indications would preferably be adapted to feed multiple renal arteries perfusing both kidneys.

In another regard, mere delivery of an agent into the natural, physiologic blood flow path of the aorta upstream of the kidneys may provide some beneficial, localized renal delivery versus other systemic delivery methods, but various undesirable results still arise. In particular, the high flow aorta immediately washes much of the delivered agent beyond the intended renal artery ostia. This reduces the amount of agent actually perfusing the renal arteries with reduced efficacy, and thus also produces unwanted loss of the agent into other organs and tissues in the systemic circulation (with highest concentrations directly flowing into downstream circulation).

In still a further regard, various known types of tubular local delivery catheters, such as angiographic catheters, other “end-hole” catheters, or otherwise, may be positioned with their distal agent perfusion ports located within the renal arteries themselves for delivering agents there, such as via a percutaneous translumenal procedure via the femoral arteries (or from other access points such as brachial arteries, etc.). However, such a technique may also provide less than completely desirable results.

For example, such seating of the delivery catheter distal tip within a renal artery may be difficult to achieve from within the large diameter/high flow aorta, and may produce harmful intimal injury within the artery. Also, where multiple kidneys must be infused with agent, multiple renal arteries must be cannulated, either sequentially with a single delivery device, or simultaneously with multiple devices. This can become unnecessarily complicated and time consuming and further compound the risk of unwanted injury from the required catheter manipulation. Moreover, multiple dye injections may be required in order to locate the renal ostia for such catheter positioning, increasing the risks associated with contrast agents on kidney function (e.g. RCN)—the very organ system to be protected by the agent delivery system in the first place. Still further, the renal arteries themselves, possibly including their ostia, may have pre-existing conditions that either prevent the ability to provide the required catheter seating, or that increase the risks associated with such mechanical intrusion. For example, the artery wall may be diseased or stenotic, such as due to atherosclerotic plaque, clot, dissection, or other injury or condition. Finally, among other additional considerations, previous disclosures have yet to describe an efficacious and safe system and method for positioning these types of local agent delivery devices at the renal arteries through a common introducer or guide sheath shared with additional medical devices used for upstream interventions, such as angiography or guide catheters. In particular, to do so concurrently with multiple delivery catheters for simultaneous infusion of multiple renal arteries would further require a guide sheath of such significant dimensions that the preferred Seldinger vascular access technique would likely not be available, instead requiring the less desirable “cut-down” technique.

In addition to the various needs for delivering agents into branch arteries described above, much benefit may also be gained from simply enhancing blood perfusion into such branches, such as by increasing the blood pressure at their ostia. In particular, such enhancement would improve a number of medical conditions related to insufficient physiological perfusion into branch vessels, and in particular from an aorta and into its branch vessels such as the renal arteries.

Certain prior disclosures have provided surgical device assemblies and methods intended to enhance blood delivery into branch arteries extending from an aorta. For example, intra-aortic balloon pumps (IABPs) have been disclosed for use in diverting blood flow into certain branch arteries. One such technique involves placing an IABP in the abdominal aorta so that the balloon is situated slightly below (proximal to) the branch arteries. The balloon is selectively inflated and deflated in a counterpulsation mode (by reference to the physiologic pressure cycle) so that increased pressure distal to the balloon directs a greater portion of blood flow into principally the branch arteries in the region of their ostia. However, the flow to lower extremities downstream from such balloon system can be severely occluded during portions of this counterpulsing cycle. Moreover, such previously disclosed systems generally lack the ability to deliver drug or agent to the branch arteries while allowing continuous and substantial downstream perfusion sufficient to prevent unwanted ischemia.

It is further noted that, despite the renal risks described in relation to radiocontrast dye delivery, and in particular RCN, in certain circumstances delivery of such dye or other diagnostic agents is indicated specifically for diagnosing the renal arteries themselves. For example, diagnosis and treatment of renal stenosis, such as due to atherosclerosis or dissection, may require dye injection into a subject renal artery. In such circumstances, enhancing the localization of the dye into the renal arteries may also be desirable. In one regard, without such localization larger volumes of dye may be required, and the dye lost into the downstream aortic flow may still be additive to impacting the kidney(s) as it circulates back there through the system. In another regard, an ability to locally deliver such dye into the renal artery from within the artery itself, such as by seating an angiography catheter there, may also be hindered by the same stenotic condition requiring the dye injection in the first place (as introduced above). Still further, patients may have stent-grafts that may prevent delivery catheter seating.

Notwithstanding the interest and advances toward delivering agents for treatment or diagnosis of organs or tissues, the previously disclosed systems and methods summarized immediately above generally lack the ability to effectively deliver agents from within a main artery and locally into substantially only branch arteries extending therefrom while allowing the passage of substantial blood flow and/or other medical devices through the main artery past the branches. This is in particular the case with previously disclosed renal treatment and diagnostic devices and methods, which do not adequately provide for local delivery of agents into the renal system from a location within the aorta while allowing substantial blood flow continuously downstream past the renal ostia and/or while allowing distal medical device assemblies to be passed retrogradedly across the renal ostia for upstream use. Much benefit would be gained if agents, such as protective or therapeutic drugs or radiopaque contrast dye, could be delivered to one or both of the renal arteries in such a manner.

Several more recently disclosed advances have included local flow assemblies using tubular members of varied diameters that divide flow within an aorta adjacent to renal artery ostia into outer and inner flow paths substantially perfusing the renal artery ostia and downstream circulation, respectively. Such disclosures further include delivering fluid agent primarily into the outer flow path for substantially localized delivery into the renal artery ostia. These disclosed systems and methods represent exciting new developments toward localized diagnosis and treatment of pre-existing conditions associated with branch vessels from main vessels in general, and with respect to renal arteries extending from abdominal aortas in particular.

However, such previously disclosed designs would still benefit from further modifications and improvements in order to: maximize mixing of a fluid agent within the entire circumference of the exterior flow path surrounding the tubular flow divider and perfusing multiple renal artery ostia; use the systems and methods for prophylaxis and protection of the renal system from harm, in particular during upstream interventional procedures; maximize the range of useful sizing for specific devices to accommodate a wide range of anatomic dimensions between patients; and optimize the construction, design, and inter-cooperation between system components for efficient, atraumatic use.

A need still exists for improved devices and methods for delivering agents principally into the renal arteries of a patient from a location within the patient's aorta adjacent the renal artery ostia along the aorta wall while at least a portion of aortic blood flow is allowed to perfuse downstream across the location of the renal artery ostia and into the patient's lower extremities.

A need still exists for improved devices and methods for substantially isolating first and second portions of aortic blood flow at a location within the aorta of a patient adjacent the renal artery ostia along the aorta wall, and directing the first portion into the renal arteries from the location within the aorta while allowing the second portion to flow across the location and downstream of the renal artery ostia into the patient's lower extremities. There is a further benefit and need for providing passive blood flow along the isolated paths and without providing active in-situ mechanical flow support to either or both of the first or second portions of aortic blood flow.

A need still exists for improved devices and methods for locally delivering agents such as radiopaque dye or drugs into a renal artery from a location within the aorta of a patient adjacent the renal artery's ostium along the aorta wall, and without requiring translumenal positioning of an agent delivery device within the renal artery itself or its ostium.

A need still exists for improved devices and methods for bilateral delivery of fluids or agents such as radiopaque dye or drugs simultaneously into multiple renal arteries feeding both kidneys of a patient using a single delivery device and without requiring translumenal positioning of multiple agent delivery devices respectively within the multiple renal arteries themselves.

A need still exists for improved devices and methods for delivery of fluids or agents into the renal arteries of a patient from a location within the patient's aorta adjacent the renal artery ostia along the aorta wall, and while allowing other treatment or diagnostic devices and systems, such as angiographic or guiding catheter devices and related systems, to be delivered across the location.

A need still exists for improved devices and methods for delivering fluids or agents into the renal arteries from a location within the aorta of a patient adjacent to the renal artery ostia along the aorta wall, and other than as a remedial measure to treat pre-existing renal conditions, and in particular for prophylaxis or diagnostic procedures related to the kidneys.

A need still exists for improved devices and methods for delivery of fluids or agents into the renal arteries of a patient in order to treat, protect, or diagnose the renal system adjunctive to performing other contemporaneous medical procedures such as angiograms other translumenal procedures upstream of the renal artery ostia.

A need still exists for improved devices and methods for delivering both an intra aortic drug delivery system and at least one adjunctive distal interventional device, such as an angiographic or guiding catheter, through a common delivery sheath.

A need also still exists for improved devices and methods for delivering both an intra aortic drug delivery system and at least one adjunctive distal interventional device, such as an angiographic or guiding catheter, through a single access site, such as a single femoral arterial puncture.

A need also still exists for improved devices and methods for treating, and in particular preventing, ARF, and in particular relation to RCN or CHF, by locally delivering renal protective or ameliorative drugs into the renal arteries, such as contemporaneous with radiocontrast injections such as during angiography procedures.

A need still exists for improved devices to deliver fluid agents bilaterally to both sides of the renal system from within the aorta system.

A need still exists for improved devices to deliver fluid agents bilaterally to both sides of the renal system without requiring cannulation of the renal arteries themselves.

A need also exists for improved devices to deliver fluid agents bilaterally to both sides of the renal system without substantially occluding, isolating, or diverting blood flow within the abdominal aorta.

In addition to these particular needs for selective fluid delivery into a patient's renal arteries via their ostia along the aorta, other similar needs also exist for fluid delivery into other branch vessels or lumens extending from other main vessels or lumens, respectively, in a patient.

### BRIEF SUMMARY OF THE INVENTION

These present embodiments therefore generally relate to intra aortic renal drug delivery systems generally from a position proximal to the renal arteries themselves; however, it is contemplated that these systems and methods may be suitably modified for use in other anatomical regions and for other medical conditions without departing from the broad scope of various of the aspects illustrated by the embodiments. For example, intra aortic fluid delivery according to various of these embodiments benefits from particular dimensions, shapes, and constructions for the subject devices herein described. However, suitable modifications may be made to deliver fluids to other multi-lateral branch structures from main body spaces or lumens, such as for example in other locations within the vasculature (e.g. right and left coronary artery ostia, fallopian tubes stemming from a uterus, or gastrointestinal tract.

One aspect of the invention is a local renal infusion system for treating a renal system in a patient from a location within the abdominal aorta associated with first and second flow paths within an outer region of abdominal aortic blood flow generally along the abdominal aorta wall and into first and second renal arteries, respectively, via their corresponding first and second renal ostia along an abdominal aorta wall in the patient. This system includes a local injection assembly with first and second injection ports. The local injection assembly is adapted to be positioned at the location with the first and second injection ports at first and second respective positions, respectively, corresponding with the first and second flow paths. The local injection assembly is also adapted to be fluidly coupled to a source of fluid agent externally of the patient when the local injection assembly is positioned at the location. Accordingly, the local injection assembly is adapted to inject a volume of fluid agent from the source, through the first and second injection ports at the first and second positions, respectively, and bi-laterally into the first and second renal arteries, also respectively. This assembly is in particular adapted to accomplish such localized bilateral renal delivery via the respective corresponding first and second renal ostia and without substantially altering abdominal aorta flow along the location.

According to certain further modes of this aspect, the local injection assembly is adapted to inject the volume of fluid agent into the first and second flow paths such that the injected volume flows substantially only into the first and second renal arteries without substantially diverting, occluding, or isolating one region of aortic blood flow with respect to the first or second regions of aortic blood flow.

Another further mode also includes a delivery member with a proximal end location and a distal end location with a longitudinal axis. The local injection assembly comprises first and second injection members with first and second injection ports, respectively, and is adapted to extend from the distal end location of the delivery member and is adjustable between a first configuration and a second configuration as follows. The local injection assembly in the first configuration is adapted to be delivered by the delivery member to the location. The local injection assembly at the location is adjustable from the first configuration to the second configuration such that the first and second first injection members are radially extended from the longitudinal axis with the first and second injection ports located at the first and second positions, respectively, at the first and second flow paths.

According to another mode, the local injection assembly includes an elongate body that is adapted to be positioned within the outer region. The first and second injection ports are spaced at different locations around the circumference of the elongate body such that the first and second injection ports are adapted to inject the volume of fluid agent in first and second different respective directions laterally from the elongate body and generally into the first and second flow paths, respectively.

According to one embodiment of this mode, a positioner cooperates with the elongate body and is adapted to position the elongate body within the outer region at the location. In one variation of this embodiment, the positioner is coupled to the elongate body and is adjustable from a first configuration to a second configuration. The positioner in the first configuration is adapted to be delivered to the location with the elongate body. The positioner at the location is adapted to be adjusted from the first configuration to the second configuration that is biased to radially extend from the elongate body relative to the first configuration and against the abdominal aorta wall with sufficient force so as to deflect the orientation of the elongate body into the outer region. In still a further embodiment, the positioner comprises a plurality of partial loop-shaped members such as described above.

In another mode of this aspect of the invention, the local injection assembly further includes an elongate body with a longitudinal axis and that is adapted to be positioned at the location. The first and second injection members in the first configuration have first radial positions relative to the longitudinal axis, and in the second configuration have second radial positions. The second radial positions are radially extended from the longitudinal axis relative to the first radial position.

In one embodiment of this mode, the first and second injection members are located on opposite respective sides of the elongate body around a circumference of the elongate body. In one variation of this embodiment, each of the first and second injection members extends between proximal and distal respective locations on each of the opposite respective sides of the elongate body, and in the second configuration the first and second injection members are biased outward from the elongate body between the respective proximal and distal respective locations.

In another embodiment, the local injection assembly is in the form of a generally loop-shaped member, such that the first and second injection members comprise first and second regions along the loop-shaped member, and whereas the first and second injection ports are located on each of the first and second regions. The loop-shaped member in the first configuration has a first diameter between the first and second injection ports such that the loop-shaped member is adapted to be delivered to the location. The loop-shaped member in the second configuration has a second diameter between the first and second injection ports that is greater than the first diameter and is sufficient such that the first and second positions generally correspond with first and second flow paths within the outer region, respectively. According to one variation of this embodiment, the local injection assembly in the second configuration for the loop-shaped member includes a memory shape. The loop-shaped member is adjustable from the second configuration to the first configuration within a radially confining outer delivery sheath. The loop-shaped member is adjustable from the first configuration to the second configuration by removing it from radial confinement outside of the outer delivery sheath.

In a further mode, first and second markers located along first and second injection members, respectively, at locations generally corresponding with the first and second injection ports. Each of the first and second markers is adapted to indicate to an operator externally of the patient the locations of the first and second injection ports to assist their delivery to the first and second positions, respectively. In particular beneficial embodiments, the first and second markers are radiopaque and provide guidance under fluoroscopy. In a further embodiment, the first and second injection members extend distally from the delivery member from a bifurcation location, and a proximal marker is located at the bifurcation location.

In another mode, a delivery member is provided that is an introducer sheath with a proximal end location and a distal end location that is adapted to be positioned at the location with the proximal end location of the introducer sheath extending externally from the patient. The delivery member includes a delivery passageway extending between a proximal port assembly along the proximal end location of the introducer sheath and a distal port at the distal end location of the introducer sheath. The injection assembly is adjustable between first and second positions. The first and second injection members are collapsed in the first longitudinal position and are extended radially from the distal end location in the second longitudinal position. In a further embodiment of this mode, the distal end location of the introducer sheath includes a distal tip and a delivery marker at a location corresponding with the distal tip such that the delivery marker is adapted to indicate the relative position of the distal tip within the abdominal aorta at the location.

In another further embodiment, a catheter body is provided with a proximal end location and a distal end location that is adapted to be positioned at the location when the proximal end location of the catheter body extends externally from the patient. The first and second injection members are coupled to and extend radially from the distal end location of the catheter body. The proximal port assembly of the introducer sheath comprises a single proximal port, and the first and second injection members and distal end location of the catheter body are adapted to be inserted into the delivery passageway through the single proximal port.

According to another mode, the system further includes a proximal coupler assembly that is adapted to be fluidly coupled to a source of fluid agent externally of the patient, and also to the first and second injection ports at the first and second positions, respectively.

In one embodiment, the proximal coupler assembly comprises first and second proximal couplers. The first proximal coupler is fluidly coupled to the first injection port, and the second proximal coupler is fluidly coupled to the second injection port. In one variation of this embodiment, a first elongate body extends between the first proximal coupler and the first injection member, and with a first fluid passageway coupled to the first proximal coupler and the first injection port; a second elongate body extends between the second proximal coupler and the second injection member, and with a second fluid passageway coupled to the second coupler and the second injection port. In another variation, the proximal coupler assembly includes a single common coupler that is fluidly coupled to each of the first and second injection ports via a common fluid passageway. According to one feature that may be employed per this variation, an elongate body extends between the single common coupler and the first and second injection members. The elongate body has at least one delivery passageway fluidly coupled to the single common coupler and also to the first and second injection ports.

According to still a further mode of this aspect of the invention, the system further includes a source of fluid agent that is adapted to be coupled to the local injection assembly. The fluid agent may comprises one, or combinations of, the following: saline; a diuretic, such as Furosemide or Thiazide; a vasopressor, such as Dopamine; a vasodilator; another vasoactive agent; Papaverine; a Calcium-channel blocker; Nifedipine; Verapamil; fenoldapam mesylate; a dopamine DA₁ agonist; or analogs or derivatives, or combinations or blends, thereof.

Another mode includes a vascular access system with an elongate tubular body with at least one lumen extending between a proximal port assembly and a distal port that is adapted to be positioned within a vessel having translumenal access to the location. The system per this mode also includes a percutaneous translumenal interventional device that is adapted to be delivered to an intervention location across the location while the local injection assembly is at the location. The local injection assembly and percutaneous translumenal interventional device are adapted to be delivered percutaneously to the location and intervention location, respectively, through the vascular access device, and are also adapted to be simultaneously engaged within the vascular access device.

In one embodiment, the percutaneous translumenal interventional device comprises an angiographic catheter. In another, the percutaneous translumenal interventional device is a guiding catheter. In another regard, the interventional device may be between about 4 French and about 8 French.

In another embodiment, the proximal port assembly includes first and second proximal ports. The percutaneous translumenal interventional device is adapted to be inserted into the elongate body through the first proximal port. The first and second ports of the injection assembly are adapted to be inserted into the elongate body through the second proximal port.

Another aspect is a local infusion system for locally delivering a volume of fluid agent from a source located externally of a patient and into a location within a body space of a patient. This system includes a delivery member with a proximal end location and a distal end location with a longitudinal axis, and a local injection assembly comprising first and second injection members with first and second injection ports, respectively. The local injection assembly extends from the distal end location of the delivery member and is adjustable between a first configuration and a second configuration as follows. The local injection assembly in the first configuration is adapted to be delivered by the delivery member to the location. The local injection assembly at the location is adjustable from the first configuration to the second configuration such that the first and second first injection members are radially extended from the longitudinal axis with the first and second injection ports located at first and second relatively unique positions, respectively, at the location. The first and second injection ports at the first and second respective positions are adapted to be fluidly coupled to a source of fluid agent externally of the patient and to inject a volume of fluid agent into the patient at the first and second positions, also respectively, at the location.

Another aspect of the invention is a local renal infusion system for treating a renal system in a patient from a location within the abdominal aorta associated with abdominal aortic blood flow into first and second renal arteries via respective first and second renal ostia having unique relative locations along the abdominal aorta wall. This system includes in one regard a delivery catheter with an elongate body having a proximal end location, a distal end location with a distal tip that is adapted to be delivered across the location and to a delivery location that is upstream of the location while the proximal end location is located externally of the patient, and a delivery lumen extending between a proximal port along the proximal end location and a distal port along the distal end location. A local injection assembly is also provided with an injection port. The local injection assembly is adapted to be delivered at least in part by the elongate body to the location such that the injection port is at a position within the location while the distal tip of the delivery catheter is at the delivery position. The injection port at the location is adapted to be fluidly coupled to a source of fluid agent located externally of the patient and to inject a volume of fluid agent from the source into abdominal aorta at the location such that the injected volume flows substantially into the first and second arteries via the first and second renal ostia, respectively.

A further aspect of the invention is a catheter for locally delivering fluid agent to the renal arteries of a patient and accommodating a medical intervention device with the catheter having a proximal end location, a mid distal location, and a distal end location and the catheter further having a central lumen and at least one outer lumen. A local injection assembly has at least one tube, wherein each tube is inserted into a corresponding outer lumen. Each tube has a proximal end location and a distal end location wherein the distal end location of each tube is coupled to the distal end location of the catheter. The local injection assembly has at least a first injection port, the injection port positioned on at least one tube between the distal end location of the tube and the mid distal location of the catheter. A fluid agent source is fluidly connected to the proximal end location of at least one tube with an injection port. Each tube is adjustable between a first position and a second position; wherein in the first position, each tube is configured to be delivered to a location within an abdominal aorta associated with a blood stream flowing into a plurality of renal artery ostia and wherein in the second position, each tube is configured to be anchored at the location and the injection port is positioned to deliver fluid agent from the fluid agent source into the blood stream. Also in the second position, the central lumen is adapted to provide a passageway from the proximal end location to the distal end location of the catheter to accommodate a medical intervention device.

In another mode of this aspect, the injection assembly has at least a second tube and at least a second injection port in the second tube.

In a further mode of this aspect, the injection assembly has at least a third tube and in a still further mode, the injection assembly has at least a fourth tube.

In a still further mode, the catheter has a longitudinal axis and the first and second tubes in the first configuration have first radial positions relative to the longitudinal axis while the first and second tubes in the second configuration have second radial positions that are radially extended from the longitudinal axis relative to the first radial position.

In another mode, the first and second tubes are located on opposite respective sides of the catheter around a circumference of the catheter.

In a further mode, each of the first and second tubes extends between the mid distal location and the distal location on each of the opposite respective sides of the catheter, and in the second configuration, the first and second tubes are biased outward from the catheter between the respective mid distal location and distal location of the catheter.

In a still further mode, first and second markers are located along the first and second tubes, respectively, at locations generally corresponding with the first and second injection ports. Each of the first and second markers is adapted to indicate to an operator externally of the patient the locations of the first and second injection ports to assist their delivery to the first and second positions, respectively.

In an embodiment of the aforementioned mode, the first and second markers are radiopaque markers.

In another mode of the invention, the first position is a memory shape for each tube and each tube is adjusted from the first position to the second position by applying an advancing force to the proximal end location of each tube in a distal direction. Further, each tube is self-adjustable from the second position to the first position with a memory recovery force upon removal of the advancing force.

Another aspect of the invention is a method for treating a renal system in a patient from a location within the abdominal aorta associated with abdominal aortic blood flow into first and second renal arteries via respective first and second renal ostia having unique relative locations along the abdominal aorta wall and performing medical intervention. This method includes in one regard delivering a delivery catheter with an elongate body and a central lumen having a proximal end location and a distal end location with a distal tip across the location and to a delivery location that is upstream of the location while the proximal end location is located externally of the patient. The method further includes delivering a local injection assembly that includes an injection port at least in part by the elongate body to the location such that the injection port is at a position within the location while the distal tip of the delivery catheter is at the delivery position. The injection port at the location is fluidly coupled to a source of fluid agent located externally of the patient. A volume of fluid agent from the source is injected through the injection port and into abdominal aorta at the location such that the injected volume flows substantially into the first and second arteries via the first and second renal ostia, respectively. Medical intervention is performed through the central lumen.

Another aspect of the invention is a method for treating a renal system in a patient from a location within the abdominal aorta associated with abdominal aortic blood flow into first and second renal arteries via their respective first and second renal ostia, respectively, at unique respective locations along the abdominal aorta wall. This method includes: positioning a local injection assembly at the location with first and second injection ports at first and second unique respective positions at the location. Also includes is fluidly coupling the local injection assembly at the location to a source of fluid agent externally of the patient. A further step includes simultaneously injecting a volume of fluid agent from the source through the first and second injection ports at the first and second positions and principally into the first and second renal arteries, respectively.

Another aspect of the invention is a method for treating a renal system in a patient from a location within the abdominal aorta associated with abdominal aortic blood flow into each of first and second renal arteries via first and second renal ostia, respectively, at unique respective locations along the abdominal aorta wall. This method includes positioning a local injection assembly at the location, and fluidly coupling to the local injection assembly at the location to a source of fluid agent externally of the patient. Also included is injecting a volume of fluid agent from the source and into the abdominal aorta at the location in a manner such that the injected fluid flows principally into the first and second renal arteries via the first and second renal ostia, respectively, and without substantially altering, occluding or isolating a substantial location of an outer region of aortic blood flow along a circumference of the abdominal aorta wall and across the location.

Another aspect of the invention is a method for treating a renal system in a patient from a location within the abdominal aorta associated with abdominal aortic blood flow into each of first and second renal arteries via first and second renal ostia, respectively, at unique respective locations along the abdominal aorta wall and performing medical intervention. This method aspect includes positioning a delivery member with a central lumen within an abdominal aorta of a patient, and delivering with the delivery member a local injection assembly having first and second injection members with first and second injection ports, respectively, in a first configuration to the location. Also included is adjusting the local injection assembly between the first configuration and a second configuration at the location. Further to this method, in the second configuration the local injection assembly extends from the distal end location of the delivery member with the first and second first injection members radially extended relative to each other across a location of the abdominal aorta at the location and with the first and second injection ports located at first and second relatively unique positions, respectively, at the location. A further mode of this aspect is fluidly coupling the first and second injection ports at the first and second respective positions to a source of fluid agent externally of the patient, and injecting a volume of fluid agent into the first and second renal arteries via their respective first and second renal ostia from the first and second positions, respectively. A still further mode is performing medical intervention through the central lumen.

Another aspect of the invention is a method for providing local therapy to a renal system in a patient from a location within the abdominal aorta associated with first and second flow paths within an outer region of abdominal aortic blood flow generally along the abdominal aorta wall and into first and second renal arteries, respectively, via their corresponding first and second renal ostia along an abdominal aorta wall in the patient. This method includes positioning a local injection assembly with a central lumen at the location with first and second injection ports at first and second respective positions, respectively, corresponding with the first and second flow paths. Also included is fluidly coupling the local injection assembly to a source of fluid agent externally of the patient when the local injection assembly is positioned at the location, and injecting a volume of fluid agent from the source, through the first and second injection ports at the first and second positions, respectively, and bilaterally into the first and second renal arteries, also respectively, via the respective corresponding first and second renal ostia without substantially occluding, isolating or altering abdominal aorta flow along the location.

Another aspect of the invention is a method for making a local renal infusion system with coronary access for treating a renal system in a patient from a location within the abdominal aorta. This method includes providing a elongated member having a central lumen and at least two outer lumens. The elongated member has a mid distal location, a distal end location, and a longitudinal axis. Each of the outer lumens has an outer wall in the elongated member. A slit of a predetermined length is made in the outer wall of each of the outer lumens parallel to the longitudinal axis of the elongated member and extending from the distal end location of the elongated member to the mid distal location of the elongated member. Single tubes to correspond with the number of outer lumens are provided where each single tube has a proximal end and a distal end. The single tubes are inserted into the corresponding outer lumens in the elongated member. The distal end of each single tube is coupled to the distal end location of the elongated member. An injection port is provided in at least two single tubes. The injection ports are positioned between the distal end of the respective single tube and the mid distal location of the elongated member. The injection ports are fluidly coupled to a source of fluid agent at the proximal end of the respective single tubes.

Further modes of these various method aspects include beneficially enhancing renal function with the injected volume of fluid agent. This may include in particular injecting the volume of fluid agent into the location while performing an interventional procedure at an intervention location within a vasculature of the patient. In one embodiment, this further includes injecting the volume of fluid agent during a period when a volume of radiocontrast dye injection is within the patient's vasculature, and such that the fluid agent is adapted to substantially prevent RCN in response to the radiocontrast dye injection. According to a further beneficial variation, the method includes treating acute renal failure with the injected volume of fluid agent.

Whereas each of these aspects, modes, embodiments, variations, and features is considered independently beneficial and are not to be required in combination with the others, nevertheless the various combinations and sub-combinations thereof as would be apparent to one of ordinary skill are further considered within the intended scope as further independently beneficial aspects of the invention.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is an anterior perspective view of an abdominal aorta in the generally vicinity of the renal arteries.

FIG. 2 is a cross-section view of an abdominal aorta taken in the vicinity of the renal arteries showing the general blood flow patterns through the abdominal aorta and the renal arteries.

FIG. 3 is a perspective view of a fluid infusion catheter in an expanded configuration.

FIG. 4 is a side plan view of the fluid infusion catheter shown in FIG. 3, and shows the fluid infusion catheter in a collapsed configuration.

FIG. 5 is a plan view of a fluid infusion catheter with positioning struts according to a further embodiment, and shows the struts in a collapsed configuration.

FIG. 6 is an anterior view of the fluid infusion catheter shown in FIG. 5, shown with the struts disposed within an abdominal aorta adjacent to the renal arteries in an expanded configuration.

FIG. 7 is a plan view of another fluid infusion catheter with struts shown in a collapsed configuration.

FIG. 8 is an anterior view of the fluid infusion catheter shown in FIG. 7, and shows the positioning struts disposed within an abdominal aorta adjacent to the renal arteries in an expanded configuration.

FIG. 9 is a plan view of another fluid infusion catheter with positioning struts shown in a collapsed configuration.

FIG. 10 is an anterior view of the fluid infusion catheter of FIG. 9, and shows the struts disposed within an abdominal aorta adjacent to the renal arteries in an expanded configuration.

FIG. 11 is a anterior view of another fluid infusion catheter with positioning loops in an extended configuration

FIG. 12 is a cross section view of the fluid infusion catheter taken at line 12-12 in FIG. 11, and shows the positioning loops in an extended configuration.

FIG. 13 is a fluid infusion catheter assembly with four positioning tubes in a collapsed state.

FIG. 14 is a cross section view of the fluid infusion catheter assembly in FIG. 13 taken at line 14-14.

FIG. 15 is the fluid infusion catheter assembly shown in FIG. 13 in an expanded state.

FIG. 16 is a cross section view of the fluid infusion catheter assembly in FIG. 15 taken at line 16-16.

FIG. 17A illustrates a proximal coupler system coupled to an embodiment of a fluid infusion catheter similar to that shown in FIG. 13.

FIG. 17B illustrates a proximal coupler system as shown in FIG. 17A with the fluid infusion catheter in an expanded state and a medical intervention device advanced into the catheter.

### DETAILED DESCRIPTION OF THE INVENTION

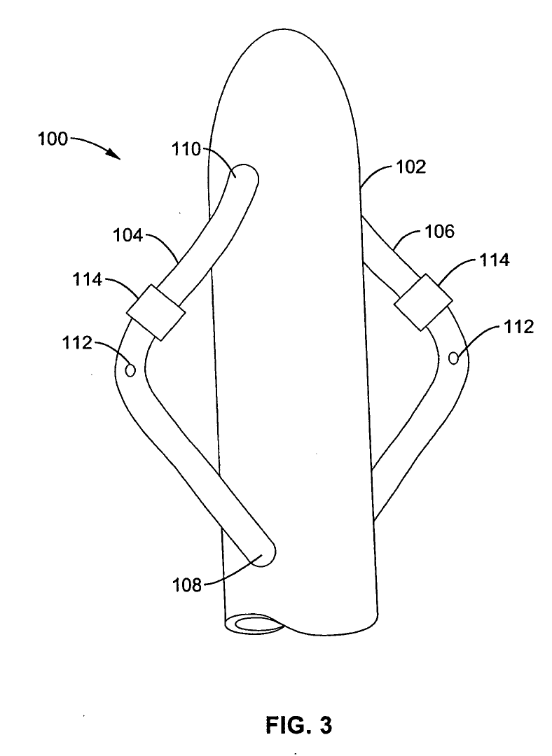

Referring more specifically to the drawings, for illustrative purposes the present invention is embodied in the apparatus generally shown in FIG. 3 through FIG. 17B. It will be appreciated that the apparatus may vary as to configuration and as to details of the parts, and that the method may vary as to the specific steps and sequence, without departing from the basic concepts as disclosed herein.

The description herein provided relates to medical material delivery systems and methods in the context of their relationship in use within a patient's anatomy. Accordingly, for the purpose of providing a clear understanding, the term proximal should be understood to mean locations on a system or device relatively closer to the operator during use, and the term distal should be understood to mean locations relatively further away from the operator during use of a system or device. These present embodiments below therefore generally relate to local renal drug delivery generally from the aorta; however, it is contemplated that these systems and methods may be suitably modified for use in other anatomical regions and for other medical conditions without departing from the broad scope of various of the aspects illustrated by the embodiments.

In general, the disclosed material delivery systems will include a fluid delivery assembly, a proximal coupler assembly and one or more elongated bodies, such as tubes or catheters. These elongated bodies may contain one or more lumens and generally consist of a proximal region, a mid-distal region, and a distal tip region. The distal tip region will typically have means for delivering a material such as a fluid agent. Radiopaque markers or other devices may be coupled to the specific regions of the elongated body to assist introduction and positioning.

The material delivery system is intended to be placed into position by a physician, typically either an interventionalist (cardiologist or radiologist) or an intensivist, a physician who specializes in the treatment of intensive-care patients. The physician will gain access to a femoral artery in the patient's groin, typically using a Seldinger technique of percutaneous vessel access or other conventional method.

For additional understanding, further more detailed examples of other systems and methods for providing local renal drug delivery are variously disclosed in the following published references: WO 00/41612 to Keren et al.; and WO 01/083016 to Keren et al. The disclosures of these references are herein incorporated in their entirety by reference thereto. Moreover, various combinations with, or modifications according to, various aspects of the present embodiments as would be apparent to one of ordinary skill upon review of this disclosure together with these references are also considered within the scope of invention as described by the various independently beneficial embodiments described below.

The invention is also related to subject matter disclosed in other Published International Patent Applications as follows: WO 00/41612 to Libra Medical Systems, published Jul. 20, 2000; and WO 01/83016 to Libra Medical Systems, published Nov. 8, 2001. The disclosures of these Published International Patent Applications are also herein incorporated in their entirety by reference thereto.

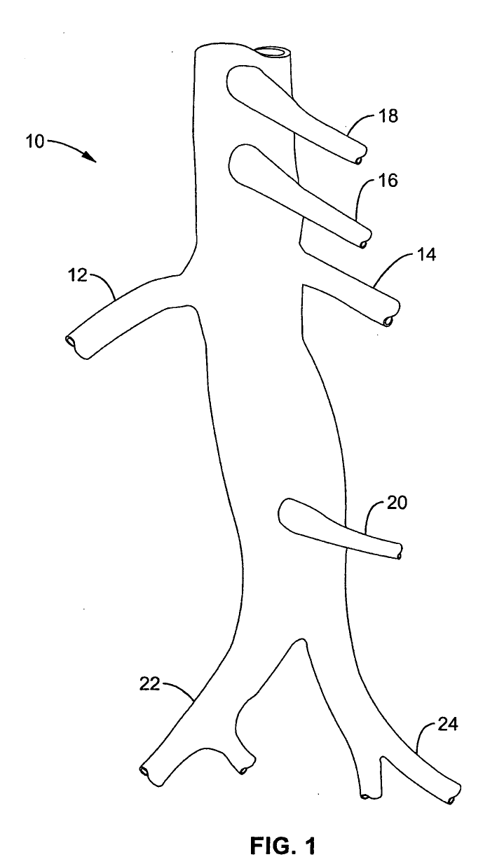

Referring initially to FIG. 1, an abdominal aorta is shown and is generally designated 10. As shown, a right renal artery 12 and a left renal artery 14 extend from the abdominal aorta 10. A superior mesenteric artery 16 extends from the abdominal aorta 10 above the renal arteries 12, 14. Moreover, a celiac artery 18 extends from the abdominal aorta 10 above the superior mesenteric artery 16. FIG. 1 also shows that an inferior mesenteric artery 20 extends from the abdominal aorta 10 below the renal arteries 12, 14. Further, as shown in FIG. 1, the abdominal aorta 10 branches into a right iliac artery 22 and a left iliac artery 24. It is to be understood that each embodiments of the present invention described in detail below can be used to deliver a drug or other fluid solution locally into the renal arteries 12, 14. Each of the below-described embodiments can be advanced through one of the iliac arteries 22, 24 and into the abdominal aorta 10 until the general vicinity of the renal arteries 12, 14 is reached.

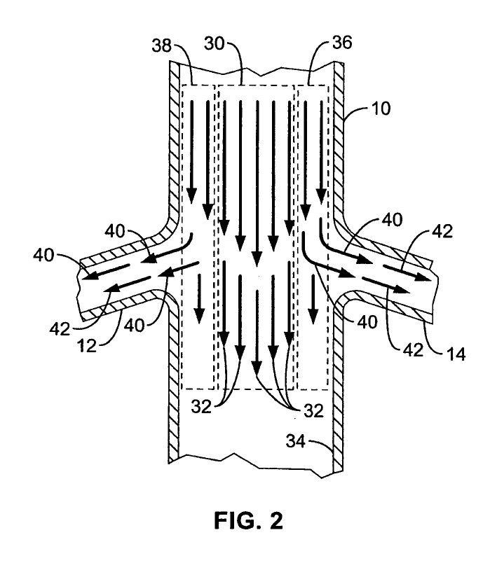

FIG. 2 shows a schematic cross-section of the abdominal aorta 10 taken in the immediate vicinity of the renal arteries 12, 14. FIG. 2 shows the natural flow patterns through the abdominal aorta 10 and the natural flow patterns from the abdominal aorta 10 into the renal arteries 12, 14. As shown, the flow down the abdominal aorta 10 maintains a laminar flow pattern. Moreover, the flow stream near the middle of the abdominal aorta 10, as indicated by dashed box 30, continues down the abdominal aorta 10, as indicated by arrows 32, and does not feed into any of the side branches, e.g., the renal arteries 12, 14. As such, a drug solution infusion down the middle of the abdominal aorta flow stream can be ineffective in obtaining isolated drug flow into the renal arteries 12, 14.

Conversely, the flow stream along an inner wall 34 of the abdominal aorta 10, as indicated by dashed box 36 and dashed box 38, contains a natural laminar flow stream into the branching arteries, e.g., the renal arteries 12, 14, as indicated by arrows 40, 42. In general, the flow stream 32 is of a higher velocity than flow stream 40 along wall 34 of aorta 10. It is to be understood that near the boundaries of dashed box 36, 38 with dashed box 30 the flow stream can contain flow streams into the branching arteries 12, 14—as well as down the abdominal aorta 10.

Further, the ostia of renal arteries 12, 14 are positioned to receive substantial blood flow from the blood flow near the posterior wall 34 of aorta 10 as well as the side walls. In other words, blood flow 40 in dashed boxes 36, 38 together is greater than blood flow 32 in dashed box 30 when along the posterior wall of aorta 10 relative to blood flow in the center of aorta 10 as shown in FIG. 2. Thus, drug infusion above renal arteries 12,14 and along the posterior wall of aorta 10 will be effective in reaching renal arteries 12, 14.

Accordingly, in order to maximize the flow of a drug solution into the renal arteries using the natural flow patterns shown in FIG. 2, it is beneficial to provide a device, as described in detail below, that is adapted to selectively infuse a drug solution along the side wall or posterior wall of the abdominal aorta 10 instead of within the middle of the abdominal aorta 10 or along the anterior wall.

As described in much greater detail below, it is beneficial to infuse a drug solution above the renal arteries 12, 14 at two locations along the wall 34 of the abdominal aorta 10 spaced approximately one-hundred and eighty degrees (180) apart from each other.

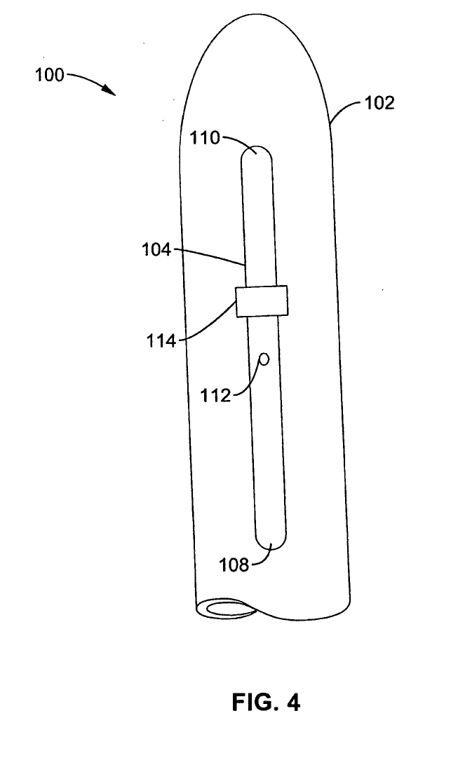

Referring now to FIG. 3 and FIG. 4, an embodiment of a drug infusion catheter is shown and is designated 100. As shown, the drug infusion catheter 100 includes a central catheter tube 102. In one beneficial embodiment, catheter tube 102 is multilumen. A first infusion tube 104 and a second infusion tube 106, made of a flexible material such as nickel-titanium tubing, are coupled to and extend from the central catheter tube 102 at approximately one-hundred and eighty degrees (180°) from each other. Each infusion tube 104, 106 includes a proximal end 108 and a distal end 110. In one beneficial embodiment, the distal ends 110 of each infusion tube 104, 106 are coupled to the central catheter tube 102 and the proximal ends 108 enter catheter tube 102 and continue proximally to a proximal coupler assembly (not shown). It is to be understood that during drug infusion, a drug solution can flow from the central catheter tube 102 and through each infusion tube 104, 106, e.g., from the proximal end 108 to the distal end 110, or from the distal end 110 to the proximal end 108, but drug solution principally exits through ports 112.

FIG. 3 and FIG. 4 show the infusion tubes 104, 106 in an expanded configuration and a retracted configuration respectively. In one embodiment, the infusion tubes 104, 106 are advanced distally from a proximal coupler assembly (not shown) causing each infusion tube 104, 106 to bow outward in the expanded configuration shown in FIG. 3. When infusion tubes 104, 106 are retracted proximally from a proximal coupler assembly (not shown), they straighten in the retracted configuration shown in FIG. 4. In another mode, infusion tubes 104, 106 are confined radially in a delivery sheath (not shown) when in a retracted configuration.

FIG. 3 and FIG. 4 further show that each infusion tube 104, 106 is formed with an infusion port 112 from which a drug solution can flow during drug infusion. Moreover, each infusion tube 104, 106 includes a marker band 114 to assist in properly positioning the catheter tube 100 within the abdominal aorta 10 (FIG. 1).

FIG. 3 shows the drug infusion catheter 100 in the expanded configuration. When expanded, the infusion tubes 104, 106 can bow away from the central catheter tube 102 in order to provide drug infusion nearer to the inner wall 34 (FIG. 1) of the abdominal aorta 10 (FIG. 1) and maintain positioning within aorta 10. When there is no longer a need for drug infusion, the infusion tubes 104, 106, are retracted against the central catheter tube 102. In the retracted configuration, shown in FIG. 4, the drug infusion catheter 100 can be inserted into the abdominal aorta 10, e.g., from the right iliac artery 22 or the left iliac artery 24. Additionally, following drug infusion, the infusion tubes 104, 106 can retract and aid in removal of the bifurcated drug infusion catheter 100 from the abdominal aorta 10 (FIG. 1).

It is to be understood that one or more additional struts or tubes (not shown) may be added to catheter 100 to position or stabilize the infusion tubes 104, 106 near the renal arteries. It is further understood that the additional struts may be made of different materials than the infusion tubes 104, 106.

In a beneficial embodiment, the drug infusion catheter 100 is used in lieu of the standard catheter introducer sheath, and its distal tip is placed at a level slightly above the renals, preferably at or below the level of the superior mesenteric artery (SMA). The drug desired to be infused selectively into the renal arteries is infused through the drug infusion catheter 100 while the coronary procedure is performed. This is a marked improvement over systemic infusion of a drug solution since the flow to the renal arteries 12, 14 is about 30 percent of total aortic blood flow.

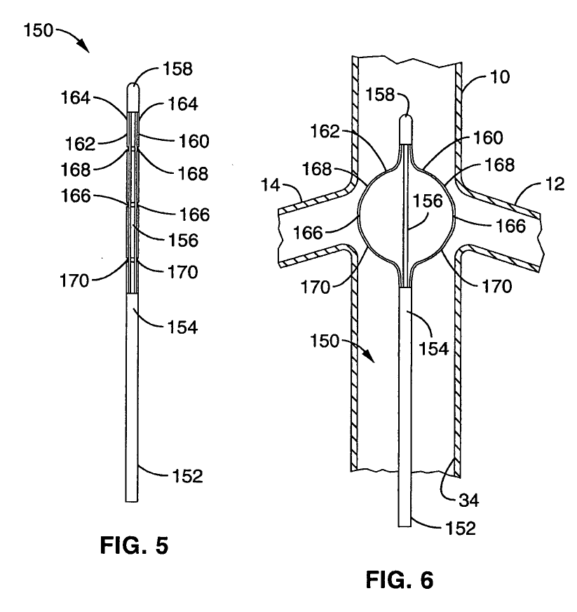

Referring now to FIG. 5 and FIG. 6, a further embodiment is a drug infusion catheter with positioning struts for positioning the catheter within an abdominal aorta is shown and is generally designated 150. FIG. 5 and FIG. 6 shows that the drug infusion catheter 150 includes an outer tube 152 that defines a proximal end (not shown) and a distal end 154. A central support tube 156 extends from within the outer tube 152 beyond the distal end 154 thereof. A tip 158 is provided at the end of the central support tube 156.

FIG. 5 and FIG. 6 show that the drug infusion catheter 150 includes a first collapsible strut 160 and a second collapsible strut 162, each in the form of a tube, and slideably disposed within the outer tube 152. Each collapsible strut 162 includes a proximal end (not shown) and a distal end 164 and the distal end 164 of each collapsible strut 162 is attached to the tip 158. As intended by the present embodiment, when each collapsible strut 160, 162 is extended out of the outer tube 152, they bow outward relative to the central support tube 156—since the distal end 164 of the strut 160, 162 is affixed to the tip 158.

As shown, each collapsible strut 160, 162 includes an infusion port 166. Further, each collapsible strut 160, 162 includes a first marker band 168 above the infusion port 166 and a second marker band 170 below the infusion port 166. Preferably, each marker band is radio-opaque to assist in positioning the drug infusion catheter 150 within the abdominal aorta 10.

FIG. 5 shows the drug infusion catheter 150 in the collapsed configuration, i.e., with the collapsible struts 160, 162 that form positioning struts in the collapsed configuration. In the collapsed configuration, the drug infusion catheter 150 can be inserted into to the right or left iliac artery 22, 24 (FIG. 1) and fed into the abdominal artery 10 until it is in proper position near the renal arteries 12, 14. Once in position near the renal arteries 12, 14, the collapsible struts 160, 162 can be advanced forward relative to the outer tube 152 causing them to release from the central support tube 156. The collapsible struts 160, 162 can be advanced forward until they establish the expanded configuration shown in FIG. 6. In the expanded configuration, the infusion ports 166 are positioned immediately adjacent to the renal arteries 12, 14 and can release a drug solution directly into the renal arteries 12, 14. It can be appreciated that the drug infusion catheter 150 can be placed so that the drug solution is infused immediately above the renal arteries 12, 14 along the wall 34 of the abdominal aorta 10. After a specified dwell time within the abdominal aorta 10, the drug infusion catheter 150 can be returned to the collapsed configuration and withdrawn from the abdominal aorta 10.

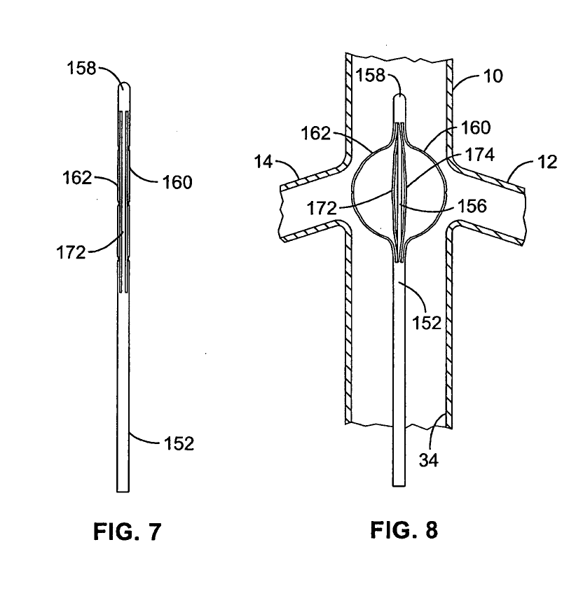

Referring briefly to FIG. 7 and FIG. 8, another embodiment of a drug infusion catheter with positioning struts is shown. FIG. 7 and FIG. 8 shows that the drug infusion catheter 150 can include a third collapsible strut 172 and/or a fourth collapsible strut 174. Accordingly, when expanded as described above, the drug infusion catheter 150 with the four collapsible struts 160, 162, 172, 174 resembles a cage. It is to be understood that collapsible struts 172, 174 can be made of different materials or may not be configured for fluid infusion.

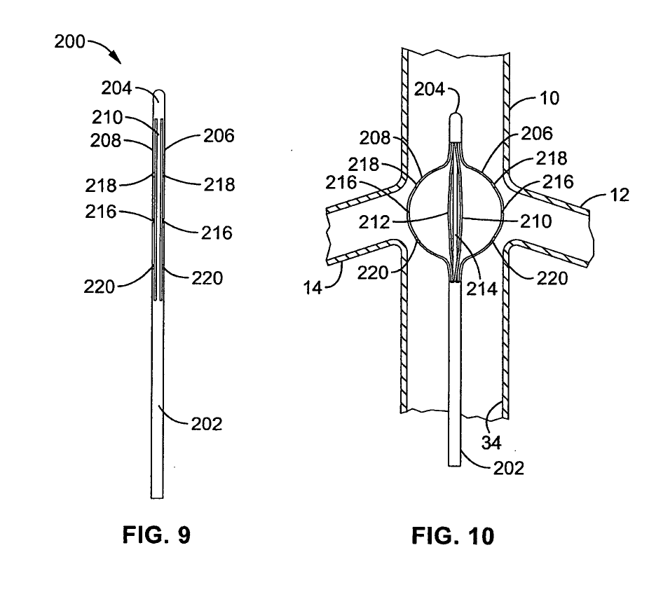

FIG. 9 and FIG. 10 show another embodiment of a drug infusion catheter with positioning struts for positioning the catheter within an abdominal aorta, generally designated 200. As shown, the drug infusion catheter 200 includes an outer tube 202 having a proximal end (not shown) and a distal end 204. A first collapsible strut 206, a second collapsible strut 208, a third collapsible strut 210, and a fourth collapsible strut 212 are established by the outer tube 202 immediately adjacent to the distal end 204 of the outer tube 202. Moreover, a central support hypotube 214 is slidably disposed within the outer tube 202. A distal end (not shown) of the central support hypotube 214 is affixed within the distal end 204 of the outer tube 202. Accordingly, as intended by the present embodiment, when the central support hypotube 214 is retracted proximally in the outer tube 202, the struts 206, 208, 210, 212 expand outward and create a cage configuration that can secure the drug infusion catheter 200, e.g., within the abdominal aorta 10 near the renal arteries 12, 14.

FIG. 9 and FIG. 10 show that the first strut 206 and the second strut 208 are each formed with an infusion port 216. Additionally, a first marker band 218 is disposed above the infusion ports 216 along each strut. And, a second marker band 220 is disposed below the infusion ports 216 along each strut. During use, a drug solution can be released from the infusion ports 216, formed in the first and second struts 206, 208. It can be appreciated that the third and/or fourth struts 210, 212 can also establish infusion ports and can further include marker bands, as described above. It can also be appreciated that drug infusion catheter 200 may be practiced with only a first and a second struts 206, 208 to present a lower profile. In a further embodiment, drug infusion catheter is practiced with first and second struts 206, 208 and a third strut 210.

FIG. 9 shows the drug infusion catheter 200 in the collapsed configuration. In the collapsed configuration, the drug infusion catheter 200 can be inserted into to the right or left iliac artery 22, 24 (FIG. 1) and fed into the abdominal artery 10 until it is in proper position near the renal arteries 12, 14. Once in position near the renal arteries 12, 14, the central support hypotube 214 is retracted proximally in outer tube 202 causing the struts 206, 208, 210, 212 to release from the central support tube 202 and bow outward. The central support hypotube 214 can be retracted proximally, as described above, until the struts 206, 208, 210, 212 establish the expanded configuration shown in FIG. 10.

In the expanded configuration, the infusion ports 216 are positioned immediately adjacent to the renal arteries 12, 14 and can release a drug solution directly into the renal arteries 12, 14. It can be appreciated that the drug infusion catheter 200 can be placed so that the drug solution is infused immediately above the renal arteries 12, 14 along the wall 34 of the abdominal aorta 10. After a specified dwell time within the abdominal aorta 10, the drug infusion catheter 200 can be returned to the collapsed configuration and withdrawn from the abdominal aorta 10.

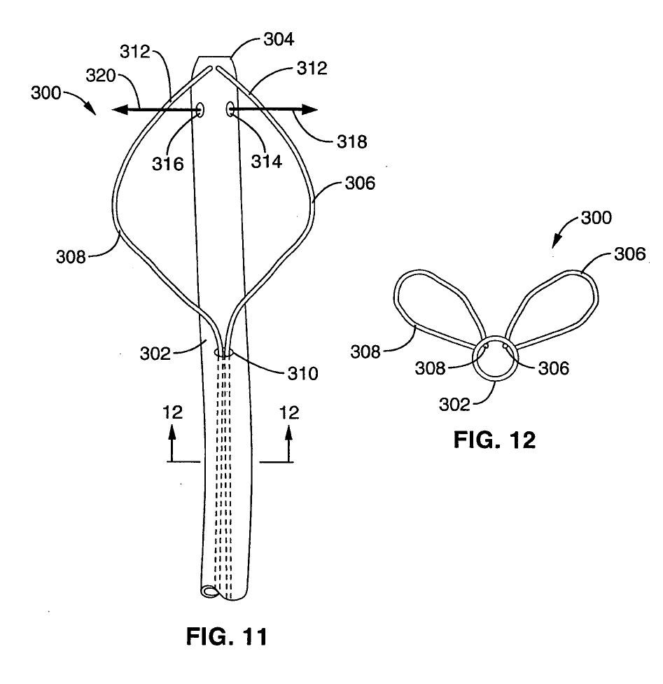

Referring to FIG. 11 and FIG. 12, another embodiment of a drug infusion catheter with positioning loops for positioning the catheter within an abdominal aorta is shown and is generally designated 300. As shown, the drug infusion catheter 300 includes a central catheter tube 302 that defines a proximal end (not shown) and a distal end 304. As shown, a first positioning wire 306 and a second positioning wire 308 extend from a port 310 formed in the central catheter tube 302. Each positioning wire 306, 308 defines a proximal end (not shown) and a distal end 312. The distal end 312 of each positioning wire 306, 308 is attached to the distal end 304 of the central catheter tube 302. It is to be understood that the positioning wires 306, 308 extend through the entire length of the central catheter tube 310 and can be used to establish an adjustable positioning loop. In one embodiment, positioning wires 306, 308 are in a separate lumen (not shown) in drug central catheter tube 302. It can be appreciated that the adjustable positioning loop can be adjusted by extending or retracting the positioning wires 306, 308 through the port 310 in the central catheter tube 302.

FIG. 11 through FIG. 12 further show that the central catheter tube 302 is formed with a first infusion port 314 and a second infusion port 316. A drug solution can exit the central catheter tube 302 and flow into the renal arteries 12, 14 as indicated by arrow 318 and 320. In a further embodiment, first and second infusion ports 314, 316 are fluidly connected to a separate lumen (not shown) in central catheter tube 302.

It can be appreciated that the drug infusion catheter 300 shown in FIG. 11 through 12 can allow rotational position adjustment and vertical position adjustment without the risk of trauma to the abdominal aorta. Further, the positioning loops 306,308 can be retracted to allow atraumatic rotation. It can be appreciated that positioning loops 306, 308 can be made of a shape-memory alloy, such as Nitinol™, and advanced through the central catheter tube 302 of catheter 300 for positioning and drug infusion, and retracted for insertion and removal.

The present embodiment recognizes that experimental observations have shown that a drug solution can flow into the renal arteries 12, 14 naturally, provided the drug infusion is undertaken above the renal arteries 12, 14 and above or closely adjacent to the posterior aspect of the inner wall 34 of the abdominal aorta 10. The positioning loops 306, 308 can easily position the central catheter tube 302 against the posterior of the inner wall 34 of the abdominal aorta 10 and does not require a flow diverter, e.g., a balloon or membrane, to maximize drug infusion to the renal arteries 12, 14. As such, the possibility of thrombus formation due to the disruption of blood flow is minimized.

It can be appreciated that the drug infusion catheter 300 can easily allow various guide catheters and guide wires to pass alongside catheter tube 302 and between positioning loops 306, 308 and that passage can have minimal effect on the tactile feedback or other performance aspects of the adjunctive catheters that are typically used in a percutaneous coronary intervention (PCI).

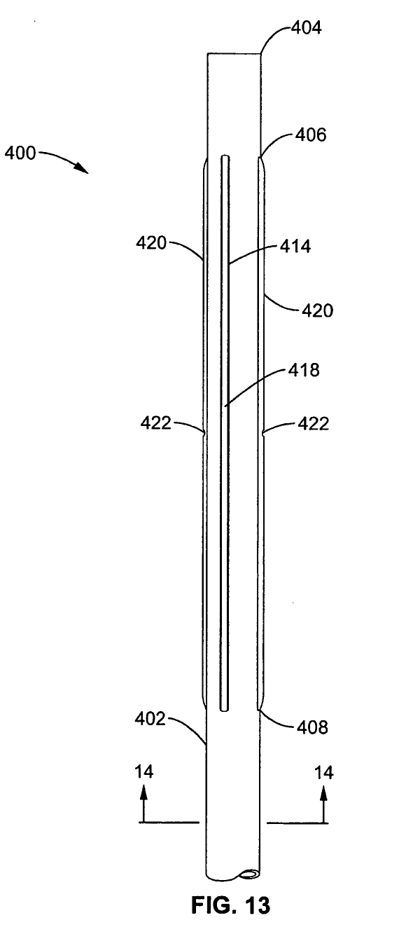

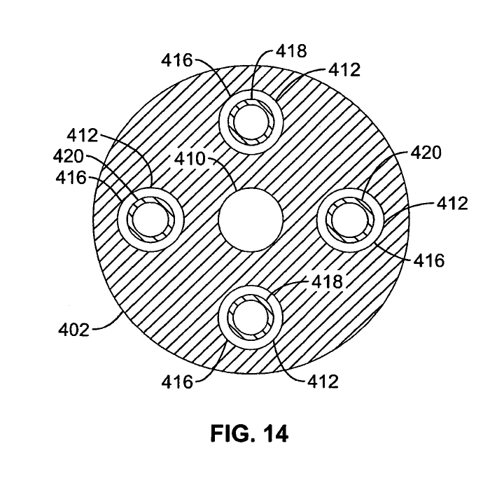

FIG. 13 through FIG. 16 illustrate another embodiment of a fluid infusion catheter assembly generally designated 400. Although intended as a fluid infusion catheter, another embodiment is used as a catheter positioning system without fluid infusion to position the catheter in a vascular location to accommodate medical intervention devices.

FIG. 13 shows multi-lumen catheter 402 has a distal tip 404, a distal location 406, a mid-distal location 408 and a proximal end (not shown). Fluid infusion catheter assembly 400 is shown in a collapsed state for insertion into the aorta and positioning near the renal arteries (see FIG. 1 and FIG. 2).

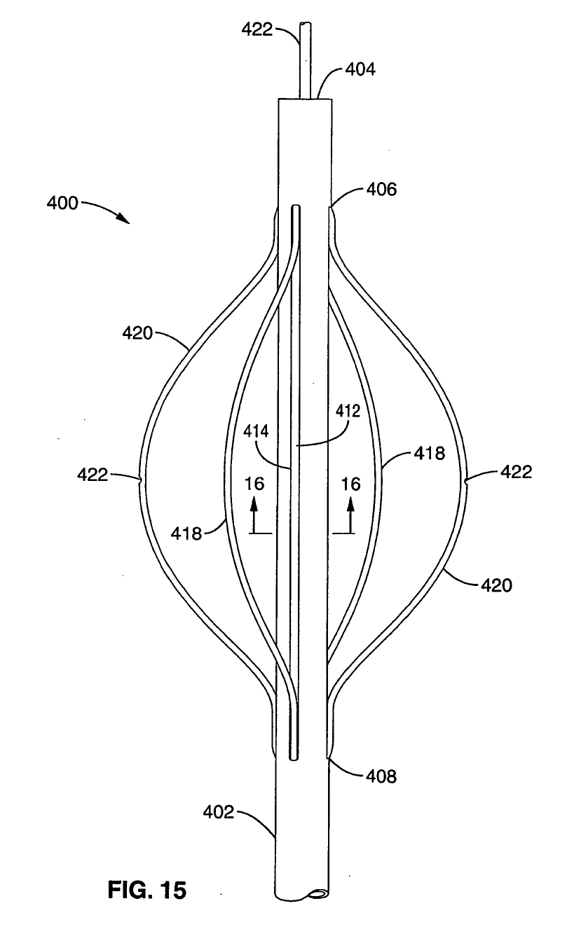

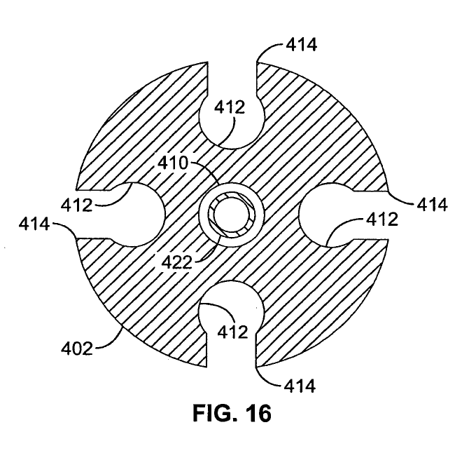

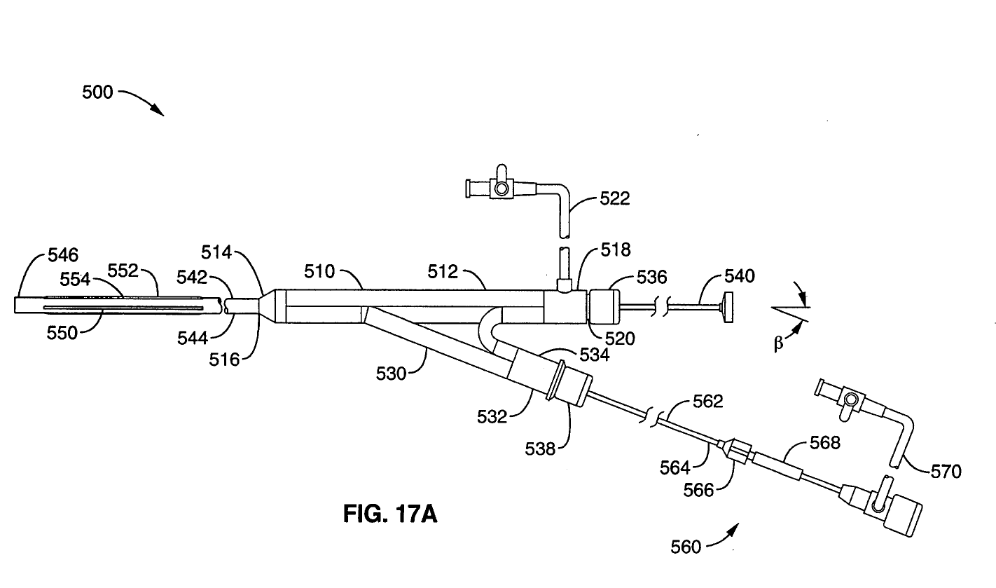

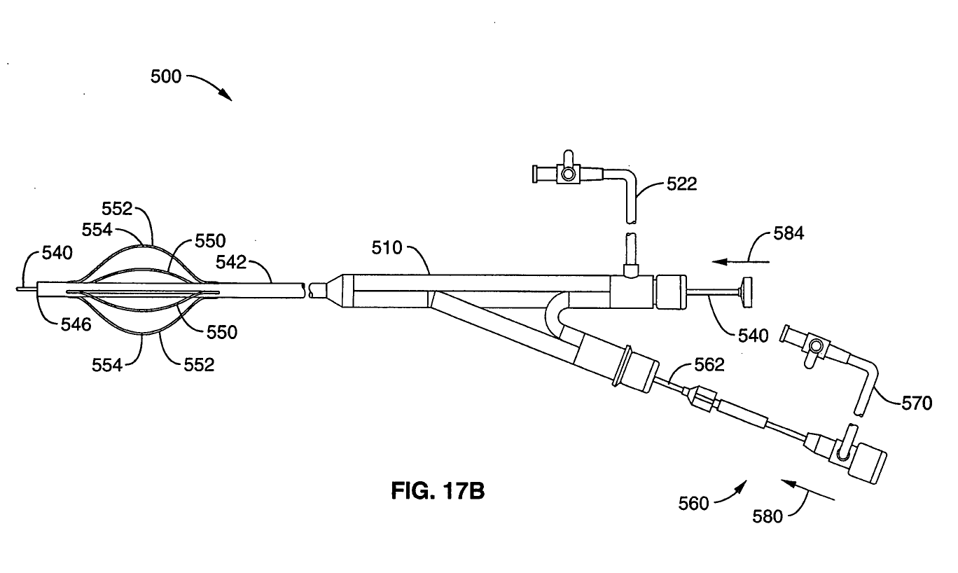

FIG. 14 is a cross section view of catheter 402 taken at line 14-14 in FIG. 13 and shows catheter 402 with a central coronary access lumen 410 and four positioning tube lumens 412. It is to be understood that the number of positioning tube lumens 412 may be different in other contemplated embodiments. Catheter 402 may be made from a polymer or other suitable material. A slit 414 is made in the outer wall 416 of each positioning tube lumen 412 in catheter 402 as shown in FIG. 13, FIG. 15 and FIG. 16. The slit may be made with a razor or another suitable cutting tool. As shown in FIG. 13, slit 414 extends from distal location 406 to mid-distal location 408. In one embodiment, slit 414 is a single cut. In another embodiment, slit 414 is at least two cuts to form a slot. Positioning tubes 418 and 420 are each inserted into positioning tube lumens 412 from the proximal end of catheter 402 (not shown) and their distal ends (not shown) coupled to catheter 402 in their respective positioning tube lumens 412 at distal location 406. Positioning tubes 418, 420 may be made from a stiff polymer, metal or other supported material. Positioning tubes 420 are shown with injection ports 422 positioned medial of distal location 406 and mid-distal location 408 of catheter 402. Positioning tubes 420 are fluidly connected to a source of fluid agent at their proximal end (not shown), typically with a proximal coupler assembly as will be described in FIG. 17A through FIG. 17B. In a further embodiment, positioning tubes 420 are positioned in adjacent positioning lumens 412. It is to be understood that positioning tubes 418 may have infusion ports in a further embodiment. It is to be further understood that positioning tubes 418 may be a solid elongated member.

In FIG. 15, positioning tubes 418, 420 are deployed by advancing their proximal ends (not shown) distally so they expand outward between distal location 406 and mid-distal location 408 of catheter 402 forming a “basket.” Positioning tubes 418, 420 place injection ports 422 at or above the renal arteries (not shown) to locally infuse fluid agent along the outer blood flow and into the renal arteries (see FIG. 2). Coronary catheter 422 is advanced distally through coronary access lumen 410 and past distal tip 404 of catheter 402 for further medical intervention. It is to be understood that other medical catheters and devices may be deployed through coronary access lumen 410.

FIG. 16 is a cross section view of catheter 402 in FIG. 15 taken at line 16-16 and illustrates a slit 414 in each positioning tube lumen 412 and coronary catheter 422 in coronary catheter lumen 410.

FIG. 17A and FIG. 17B illustrates a proximal coupler system 500 used to deploy and position renal fluid delivery devices adjunctive with interventional catheters. Y Hub body 510 has main branch 512 with a catheter fitting 514 at the distal end 516 of hub body 510 and a main adapter fitting 518 at the proximal end 520 of Y hub body 510. Main branch fluidly connects catheter fitting 514 and main port 518. By way of example and not of limitation, one embodiment of main branch 512 is adapted to accommodate a 6 Fr guide catheter. Side port fitting 522 is positioned on main branch 512 and is fluidly connected to main branch 512 to provide fluids into main branch 512 during use. Secondary branch 530 intersects main channel 512 at predetermined transition angle β. In one beneficial embodiment, transition angle β is approximately 20 degrees. Secondary branch has secondary port 532 at proximal end 534 of secondary branch 530. Y hub body 510 may be molded in one piece or assembled from a plurality of pieces.

A hemostasis valve 536 is attached at main port 518 and a Touhy Borst valve 538 is attached at secondary branch port 532. An intervention catheter 540 is introduced into the Y hub 510 through hemostasis valve 536. A multilumen fluid infusion catheter 542, similar to that shown in FIG. 15, with proximal end 544 and distal end 546, is coupled to Y hub body 510 with proximal end 544 at catheter fitting 514. Fluid infusion catheter 542 has a plurality of positioning tubes 550 and infusion tubes 552 in fluid infusion catheter 542. Infusion tubes 552 have injection ports 554 as previously described in FIG. 13 through FIG. 16. Y hub body 510 is coupled to a local fluid delivery system 560. A stiff tube 562, passes through secondary branch port 532 and Touhy Borst valve 538 and is physically and fluidly connected to infusion tubes 552 and physically connected to positioning tubes 550. In one embodiment, positioning tubes 550 and infusion tubes 552 are fluidly and physically joined to stiff tube 562 in secondary branch 530. In another embodiment, stiff tube 562 is made of a Nickel-Titanium alloy. At proximal end 564 of stiff tube 562 a handle 566 is attached. A fluid injection coupling 568 is fluidly connected to the proximal end 564 of stiff tube 562. Fluid injection system 570 is coupled to fluid injection port 568 for introducing materials such as fluids. Details of fluid injection system 570 are omitted here for clarity. In one aspect of the invention, Y hub 510, fluid delivery system 560, and fluid infusion catheter 542 are provided as a kit.

In FIG. 17B, distal end 546 of fluid infusion catheter 542 is positioned upstream of the renal arteries (not shown) with an introducer sheath, a dilator, a guide wire or other known vascular positioning method. Local fluid delivery system 560 is pushed into secondary port 532 of Y hub 510 as shown by arrow 580. Stiff tube 562 is advanced through fluid infusion catheter 542 and causes positioning tubes 550 and infusion tubes 552 to bow outward to anchor against the aortic wall (see FIG. 1 and FIG. 2). This step may be repeated if fluid infusion catheter 542 requires further alignment in relation to the renal arteries. Fluid injection system 570 injects fluid into fluid delivery system 560 which flows out of injection ports 554 in infusion tubes 552 (as previously described in FIG. 15). Arrow 584 denotes that intervention catheter 540 is advanced through the main branch 512 of Y hub assembly 510 and through the center lumen of fluid infusion catheter 542 and out the distal end 546 of fluid infusion catheter 542 to perform medical intervention procedures.

In one embodiment, the distal end 546 of fluid infusion catheter 542 is a truncated cone shape (not shown). In one mode of this embodiment, fluid infusion catheter 542 is adapted to accommodate a dilator. In another embodiment one or more radiopaque marker bands (not shown) are attached at the distal end 546 of fluid infusion catheter 542. In a further embodiment one or more radiopaque marker bands (not shown) are attached to positioning tubes 550 and/or infusion tubes 552. In a still further embodiment, infusion tubes 552 and/or positioning tubes 550 are used to position the distal end 546 of catheter 542 without fluid infusion.

In another embodiment, fluid infusion catheter 542 is introduced into the vascular system through an introducer sheath (not shown). By way of example and not of limitation, proximal coupler system 500 may be adapted to advance a wide mix of medical devices such as guide wires, diagnostic catheters, flow diverters and infusion assemblies through fluid infusion catheter 542 and into a vascular system such as aorta system 10. A multiple Y proximal coupler (not shown), with two or more branch ports, can be used to control multiple positioning tubes and infusion tubes or advance multiple medical devices.

It is to be understood that each of the embodiments described in detail above provide a device that can be used for selective therapeutic drug infusion at sites remote to a primary treatment site. These devices can be applicable to interventional radiology procedures, including interventional diagnostic and therapeutic procedures involving the coronary arteries. Further, each of the devices described above, can be beneficial for delivering certain drugs, e.g., papaverine; Nifedipine; Verapamil; fenoldopam mesylate; Furosamide; Thiazide; and Dopamine; or analogs, derivatives, combinations, or blends thereof, to the renal arteries of a patient who is simultaneously undergoing a coronary intervention with the-intent of increasing the kidney's ability to process of organically-bound iodine, i.e., radiographic contrast, as measured by serum creatinine and glomerular filtration rate (GFR).

The various embodiments herein described for the present invention can be useful in treatments and therapies directed at the kidneys such as the prevention of radiocontrast nephropathy (RCN) from diagnostic treatments using iodinated contrast materials. As a prophylactic treatment method for patients undergoing interventional procedures that have been identified as being at elevated risk for developing RCN, a series of treatment schemes have been developed based upon local therapeutic agent delivery to the kidneys. Among the agents identified for such treatment are normal saline (NS) and the vasodilators papaverine (PAP) and fenoldopam mesylate (FM).

The approved use for fenoldopam is for the in-hospital intravenous treatment of hypertension when rapid, but quickly reversible, blood pressure lowering is needed. Fenoldopam causes dose-dependent renal vasodilation at systemic doses as low as approximately 0.01 mcg/kg/min through approximately 0.5 mcg/kg/min IV and it increases blood flow both to the renal cortex and to the renal medulla. Due to this physiology, fenoldopam may be utilized for protection of the kidneys from ischemic insults such as high-risk surgical procedures and contrast nephropathy. Dosing from approximately 0.01 to approximately 3.2 mcg/kg/min is considered suitable for most applications of the present embodiments, or about 0.005 to about 1.6 mcg/kg/min per renal artery (or per kidney). As before, it is likely beneficial in many instances to pick a starting dose and titrate up or down as required to determine a patient's maximum tolerated systemic dose. Recent data, however, suggest that about 0.2 mcg/kg/min of fenoldopam has greater efficacy than about 0.1 mcg/kg/min in preventing contrast nephropathy and this dose is preferred.

The dose level of normal saline delivered bilaterally to the renal arteries may be set empirically, or beneficially customized such that it is determined by titration. The catheter or infusion pump design may provide practical limitations to the amount of fluid that can be delivered; however, it would be desired to give as much as possible, and is contemplated that levels up to about 2 liters per hour (about 25 cc/kg/hr in an average about 180 lb patient) or about one liter or 12.5 cc/kg per hour per kidney may be beneficial.

Local dosing of papaverine of up to about 4 mg/min through the bilateral catheter, or up to about 2 mg/min has been demonstrated safety in animal studies, and local renal doses to the catheter of about 2 mg/min and about 3 mg/min have been shown to increase renal blood flow rates in human subjects, or about 1 mg/min to about 1.5 mg/min per artery or kidney. It is thus believed that local bilateral renal delivery of papaverine will help to reduce the risk of RCN in patients with pre-existing risk factors such as high baseline serum creatinine, diabetes mellitus, or other demonstration of compromised kidney function.

It is also contemplated according to further embodiments that a very low, systemic dose of papaverine may be given, either alone or in conjunction with other medical management such as for example saline loading, prior to the anticipated contrast insult. Such a dose may be on the order for example of between about 3 to about 14 mg/hr (based on bolus indications of approximately 10-40 mg about every 3 hours—papaverine is not generally dosed by weight). In an alternative embodiment, a dosing of 2-3 mg/min or 120-180 mg/hr. Again, in the context of local bilateral delivery, these are considered halved regarding the dose rates for each artery itself.

Notwithstanding the particular benefit of this dosing range for each of the aforementioned compounds, it is also believed that higher doses delivered locally would be safe. Titration is a further mechanism believed to provide the ability to test for tolerance to higher doses. In addition, it is contemplated that the described therapeutic doses can be delivered alone or in conjunction with systemic treatments such as intravenous saline.

From the foregoing discussion, it will be appreciated that the various embodiments described herein generally provide for infusion of renal protective drugs into each of two renal arteries perfusing both kidneys in a patient. The devices and methods of these embodiments are useful in prophylaxis or treatment of kidney malfunction or conditions, such as for example ARF. Various drugs may be delivered via the systems and methods described, including for example: vasodilators; vasopressors; diuretics; Calcium-channel blockers; or dopamine DA1 agonists; or combinations or blends thereof. Further, more specific, examples of drugs that are contemplated in the overall systems and methods described include but are not limited to: Papaverine; Nifedipine; Verapamil; Fenoldapam; Furosamide; Thiazide; and Dopamine; or analogs, derivatives, combinations, or blends thereof.

It is to be understood that the invention can be practiced in other embodiments that may be highly beneficial and provide certain advantages. For example radiopaque markers are shown and described above for use with fluoroscopy to manipulate and position the introducer sheath and the intra aortic catheters. The required fluoroscopy equipment and auxiliary equipment devices are typically located in a specialized location limiting the in vivo use of the invention to that location. Other modalities for positioning intra aortic catheters are highly beneficial to overcome limitations of fluoroscopy. For example, non-fluoroscopy guided technology is highly beneficial for use in operating rooms, intensive care units, and emergency rooms, where fluoroscopy may not be readily available or its use may cause undue radiation exposure to users and others due to a lack of specific radiation safeguards normally present in angiography suites and the like. The use of non-fluoroscopy positioning allows intra aortic catheter systems and methods to be used to treat other diseases such as ATN and CHF in clinical settings outside of the angiography suite or catheter lab.

In one embodiment, the intra aortic catheter is modified to incorporate marker bands with metals that are visible with ultrasound technology. The ultrasonic sensors are placed outside the body surface to obtain a view. In one variation, a portable, noninvasive ultrasound instrument is placed on the surface of the body and moved around to locate the device and location of both renal ostia. This technology is used to view the aorta, both renal ostia and the intra aortic catheter.

In another beneficial embodiment, ultrasound sensors are placed on the introducer sheath and the intra aortic catheter itself; specifically the tip of the aortic catheter or at a proximal section of the catheter. The intra aorta catheter with the ultrasonic sensors implemented allows the physician to move the sensors up and down the aorta to locate both renal ostia.

A further embodiment incorporates Doppler ultrasonography with the intra aortic catheters. Doppler ultrasonography detects the direction, velocity, and turbulence of blood flow. Since the renal arteries are isolated along the aorta, the resulting velocity and turbulence is used to locate both renal ostia. A further advantage of Doppler ultrasonography is it is non-invasive and uses no x rays.

A still further embodiment incorporates optical technology with the intra aorta catheter. An optical sensor is placed at the tip of the introducer sheath. The introducer sheath's optical sensor allows visualization of the area around the tip of the introducer sheath to locate the renal ostia. In a further mode of this embodiment, a transparent balloon is positioned around the distal tip of the introducer sheath. The balloon is inflated to allow optical visual confirmation of renal ostium. The balloon allows for distance between the tip of the introducer sheath and optic sensor while separating aorta blood flow. That distance enhances the ability to visualize the image within the aorta. In a further mode, the balloon is adapted to allow profusion through the balloon wall while maintaining contact with the aorta wall. An advantage of allowing wall contact is the balloon can be inflated near the renal ostium to be visually seen with the optic sensor. In another mode, the optic sensor is placed at the distal tips of the intra aortic catheter. Once the intra aortic catheter is deployed within the aorta, the optic sensor allows visual confirmation of the walls of the aorta. The intra aortic catheter is tracked up and down the aorta until visual confirmation of the renal ostia is found. With the optic image provided by this mode, the physician can then track the positioning of the intra aortic catheter to the renal arteries.

Another embodiment uses sensors that measure pressure, velocity, and/or flow rate to locate renal ostia without the requirement of fluoroscopy equipment. The sensors are positioned at the distal end of the intra aortic catheter. The sensors display real time data about the pressure, velocity, and/or flow rate. With the real-time data provided, the physician locates both renal ostia by observing the sensor data when the intra aortic catheter is around the approximate location of the renal ostia. In a further mode of this embodiment, the intra aortic catheter has multiple sensors positioned at a mid distal and a mid proximal position on the catheter to obtain mid proximal and mid distal sensor data. From this real time data, the physician can observe a significant flow rate differential above and below the renal arteries and locate the approximate location. With the renal arteries being the only significant sized vessels within the region, the sensors would detect significant changes in any of the sensor parameters.

In a still further embodiment, chemical sensors are positioned on the intra aortic catheter to detect any change in blood chemistry that indicates to the physician the location of the renal ostia. Chemical sensors are positioned at multiple locations on the intra aortic catheter to detect chemical change from one sensor location to another.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1.-43. (canceled)

44. A catheter for locally delivering fluid agent to the renal arteries of a patient and accommodating a medical intervention device comprising:
a catheter having a proximal end location, a mid distal location, a distal end location, a central lumen that accommodates the medical interventional device, and at least one outer lumen;
a local injection assembly having a tube, wherein the tube is inserted into the outer lumen, the tube having a proximal end location, a distal end location, and a first injection port positioned on the tube between the distal end location of the tube and the mid distal location of the catheter, wherein the distal end location of the tube is coupled to the distal end location of the catheter, and the tube is adjustable between a first position and a second position;
wherein in the first position, the tube is configured to be delivered to a location within an abdominal aorta associated with a blood stream flowing into a plurality of renal artery ostia; and
wherein in the second position, the tube is configured to be anchored at the location and the injection port is positioned to deliver fluid agent from a fluid agent source into the blood stream.

45. The catheter according to claim 44, further comprising a fluid agent source fluidly connected to the proximal end location of the tube.

46. The catheter according to claim 44, wherein the central lumen is adapted to provide a passageway from the proximal end location to the distal end location of the catheter to accommodate a medical intervention device.

47. The catheter according to claim 44, further comprising:
at least a second tube; and
at least a second injection port positioned in the second tube.

48. The catheter according to claim 47, wherein:
the catheter has a longitudinal axis;
the first and second tubes in the first configuration have first radial positions relative to the longitudinal axis; and
the first and second tubes in the second configuration have second radial positions that are radially extended from the longitudinal axis relative to the first radial position.

49. The catheter according to claim 47, wherein:
the first and second tubes are located on opposite respective sides of the catheter around a circumference of the catheter.

50. The catheter according to claim 47, wherein:
each of the first and second tubes extends between the mid distal location and the distal location on each of the opposite respective sides of the catheter; and
in the second configuration the first and second tubes are biased outward from the catheter between the respective mid distal location and distal location of the catheter.

51. The catheter according to claim 47, further comprising:
first and second markers located along the first and second tubes, respectively, at locations generally corresponding with the first and second injection ports; and
wherein each of the first and second markers is adapted to indicate to an operator externally of the patient the locations of the first and second injection ports to assist their delivery to the first and second positions, respectively.

52. The catheter according to claim 51, wherein the first and second markers comprise radiopaque markers.

53. The catheter according to claim 44, wherein:
the first position is a memory shape for the tube;
the tube is adjusted from the first position to the second position by applying an advancing force to the proximal end location of the tube in a distal direction; and
the tube is self-adjustable from the second position to the first position with a memory recovery force upon removal of the advancing force.

54. A method for treating a renal system in a patient from a location within the abdominal aorta associated with abdominal aortic blood flow into first and second renal arteries via their respective first and second renal ostia, respectively, at unique respective locations along the abdominal aorta wall, and performing medical intervention, comprising:
positioning a local injection assembly with a central lumen at the location with first and second injection ports at first and second unique respective positions at the location;
fluidly coupling the local injection assembly at the location to a source of fluid agent externally of the patient;
simultaneously injecting a volume of fluid agent from the source through the first and second injection ports at the first and second positions and principally into the first and second renal arteries, respectively, and
advancing a medical intervention device through the central lumen.

55. The method according to claim 54, further comprising:
enhancing renal function with the injected volume of fluid agent.

56. The method according to claim 55, further comprising:
injecting the volume of fluid agent during a period when a volume of radiocontrast dye injection is within the patient's vasculature, wherein the fluid agent is adapted to substantially prevent RCN in response to the radiocontrast dye injection.

57. The method according to claim 55, further comprising:
treating acute renal failure with the injected volume of fluid agent.

58. The method according to claim 55, further comprising:
providing an elongated member, the elongated member having a central lumen and a first outer lumen and at least a second outer lumen;
wherein the elongated member further having a mid distal location, a distal end location, and a longitudinal axis;
wherein each of the outer lumens having an outer wall in the elongated member;
making a slit of a predetermined length in the outer wall of each outer lumen, the slit made parallel to the longitudinal axis of the elongated member and extending from the distal end location of the elongated member to the mid distal location of the elongated member;
providing a first single tube and at least a second single tube, each single tube with a proximal end and a distal end;
inserting the single tubes into the corresponding outer lumens in the elongated member;
coupling the distal end of each single tubes to the distal end location of the elongated member;
placing the first injection port on the first single tube;
placing the second injection port on the second single tube; and
positioning the first injection port and the second injection port between the distal end of the first and second single tubes respectively and the mid distal location of the elongated member.

59. The method according to claim 58, further comprising providing a third outer lumen and a third single tube.

60. The method according to claim 59, further comprising providing at least a fourth outer lumen and at least a fourth single tube.

61. A local renal infusion system for treating a renal system in a patient from a location within the abdominal aorta associated with first and second flow paths within an outer region of abdominal aortic blood flow generally along the abdominal aorta wall and into first and second renal arteries, respectively, via their corresponding first and second renal ostia along an abdominal aorta wall in the patient, comprising:
an elongated member, the elongated member having a proximal end location, a mid distal location, a distal end location, and a longitudinal axis;
the elongated member further having a central lumen, a first outer lumen and at least a second outer lumen;
each of the outer lumens having an outer wall in the elongated member;
a slit of a predetermined length in the outer wall of each outer lumen, the slit made parallel to the longitudinal axis of the elongated member and extending from the distal end location of the elongated member to the mid distal location of the elongated member;
a local injection assembly with a first single tube and at least a second single tube, wherein each single tube is inserted into a corresponding outer lumen;
each single tube having a proximal end and a distal end;
wherein the distal end of each single tube is coupled to the distal end location of the elongated member;
the first single tube having a first injection port positioned between the distal end of the first single tube and the mid distal location of the elongated member;
the second single tube having a second injection port positioned between the distal end of the second single tube and the mid distal location of the elongated member;
the single tubes adjustable between a first configuration and a second configuration;
the single tubes radially collapsed relative to the longitudinal axis of the elongated member in the first configuration;
wherein in the second configuration, the single tubes extend radially from the longitudinal axis of the elongated member through the slit in the outer walls when the proximal ends of the single tubes are advanced distally;
wherein in the second configuration, the first port and second port are at a first position and a second position respectively;
wherein the local injection assembly is adapted to be positioned at the location with the first and second injection ports at first and second positions, respectively, corresponding with the first and second flow paths;
wherein the local injection assembly is adapted to be fluidly coupled to a source of fluid agent externally of the patient when the local injection assembly is positioned at the location; and
wherein the local injection assembly is adapted to inject a volume of fluid agent from the source, through the first and second injection ports at the first and second positions, respectively, and bi-laterally into the first and second renal arteries, also respectively, via the respective corresponding first and second renal ostia without substantially altering abdominal aorta flow along the location.

62. The system according to claim 61, wherein the local injection assembly is adapted to inject the volume of fluid agent into the first and second flow paths such that the injected volume flows substantially only into the first and second renal arteries without substantially diverting one region of aortic blood flow into another region of aortic blood flow.

63. The system according to claim 61, wherein the local injection assembly is adapted to inject the volume of fluid agent into the first and second flow paths such that the injected volume flows substantially only into the first and second renal arteries without substantially occluding abdominal aortic blood flow.
